(19) United States
(12) Patent Application Publication
(10) Pub. No.: US 2026/0256378 A1
(43) Pub. Date: Sep. 3, 2026

(54) DEVICE AND METHOD FOR MEASURING METABOLIC RATE USING SEQUENTIAL GAS DELIVERY

(71) Applicant: THORNHILL SCIENTIFIC INC., North York (CA)

(72) Inventors: Joseph Arnold FISHER, Thornhill (CA); James DUFFIN, Toronto (CA); Olivia SOBCZYK, Etobicoke (CA); Bryan Drew MILLER, Richmond Hill (CA); Rafay KHAN, Markham (CA)

(21) Appl. No.: 19/149,087

(22) PCT Filed: Mar. 14, 2024
(86) PCT No.: PCT/IB2024/052482

§ 371 Date: Jul. 17, 2025

Related U.S. Application Data
(60) Provisional application No. 63/451,973, filed on Mar. 14, 2023.

(51) Int. Cl. A61B 5/083; A61B 5/091

(52) U.S. Cl. CPC A61B 5/0833; A61B 5/0836; A61B 5/091; A61B 2560/0257

(57) ABSTRACT

The present disclosure provides a device and method for measuring metabolic rate using sequential gas delivery. By delivering a first gas with a known quantity of gas X and a volume smaller than the subject's alveolar volume, and subsequently delivering a second gas to the subject, the metabolic rate can be accurately and precisely determined. The device measures an exhaled partial pressure of gas X and computes the metabolic rate based on the inhaled partial pressure of gas X, the exhale partial pressure of gas X, and the volume of the first gas.

### CROSS-REFERENCE TO RELATED APPLICATION(S)

This application claims the benefit of U.S. Provisional Patent Application No. 63/451,973 entitled “SIMPLIFIED METHOD TO MEASURE BASAL OXYGEN CONSUMPTION AND CARBON DIOXIDE PRODUCTION”, filed Mar. 14, 2023, the entire contents of which are incorporated herein by reference.

### FIELD

The present specification is directed to metabolic analysis, and particularly a device and method for measuring metabolic rate.

### BACKGROUND

The measurement of expired carbon dioxide (CO₂) and oxygen (O₂) levels during respiration presents various challenges in accurately determining the volumes of gas exchanged. The transition from the anatomical dead space to alveolar gas during exhalation makes it difficult to measure the partial pressure of CO₂ (PCO₂) and the expired flow rate accurately, leading to uncertainties in the expired volume of CO₂ (FE CO₂) and, consequently, in the volume of CO₂ consumed (VĊO₂). Additionally, the functional residual capacity (FRC), which is the volume remaining in the alveoli at the end of exhalation, is unknown, further complicating accurate measurements. These measurements are integrated crudely, and variables like different time constants for CO₂ concentration and flow exacerbate the inaccuracy, making precise CO₂ volume calculation elusive over individual breaths, although they may approximate accuracy over many breaths.

Similarly, measuring oxygen consumption (\dot{V}O₂) involves its own set of challenges, including the need for the subject to remain completely at rest to avoid inaccuracies caused by physical activity, which affects CO₂ production and O₂ consumption. Any movement, muscle tension, or deviation from normal temperature and breathing patterns can skew measurements. Furthermore, technological limitations such as the response time and accuracy of flowmeters and O₂ sensors add to the difficulty of precisely measuring \dot{V}O₂. The inherent variability in anatomical dead space among individuals and the challenges in integrating flow and gas concentration data result in cumulative errors in calculating gas volumes on a breath-by-breath basis, thus affecting the overall accuracy of these respiratory measurements.

### SUMMARY

An aspect of the specification provides a method of measuring a subject's metabolic rate using sequential gas delivery. The method includes delivering a first gas over a first portion of the subject's inhalation. The first gas has an inhaled partial pressure of gas X, and the volume of the first gas is equal to or less than the subject's alveolar volume. The method further includes delivering a second gas over a second portion of the subject's inhalation. The second gas has a partial pressure of gas X equal to a partial pressure of gas X exhaled in a prior breath. The method further includes measuring an exhaled partial pressure of gas X during the subject's exhalation and computing the metabolic rate based on the inhaled partial pressure of gas X, the exhaled partial pressure of gas X, and the volume of the first gas.

In some examples, the method further includes measuring the subject's tidal volume over a series of breaths, calculating a mean tidal volume, and estimating the subject's alveolar volume as the mean tidal volume minus anatomical dead space.

In further examples, the volume of the first gas is computed as about 70% of the estimated alveolar volume.

In further examples, the method includes identifying a subset of breaths corresponding to a steady state. The subset of breaths is identified by comparing a measured characteristic for the subset of breaths to a threshold of variability and selecting breaths for which the characteristic is below the threshold of variability. The mean tidal volume is calculated by averaging the tidal volume for the subset of breaths.

In further examples, the method includes measuring the barometric pressure and computing the metabolic rate based on the barometric pressure.

In further examples, computing the metabolic rate comprises computing

$$\dot{V} X = VA \frac{PETX - PIX}{PB} ,$$

wherein \dot{V}X represents the metabolic rate of gas X, VA represents the alveolar ventilation, PETX represents the end tidal partial pressure of gas X, PIX represents the inhaled partial pressure of gas X, and PB represents the barometric pressure.

In further examples, gas X comprises oxygen and computing the metabolic rate comprises computing the oxygen consumption rate (\dot{V}O₂).

In further examples, gas X comprises carbon dioxide and computing the metabolic rate comprises computing the carbon dioxide production rate (\dot{V}CO₂).

In further examples, gas X comprises both carbon dioxide and oxygen and computing the metabolic rate comprises computing both \dot{V}CO₂ and \dot{V}O₂.

A further aspect of the specification provides a non-transitory computer-readable medium comprising instructions that, when executed by a processor, perform the above-described method.

A further aspect of the specification provides a device for measuring a subject's metabolic rate. The device includes a gas blender and a processor connected to the gas blender. The processor is configured to control the gas blender to deliver a first gas over a first portion of the subject's inhalation. The first gas has an inhaled partial pressure of gas X, and the volume of the first gas is equal to or less than the subject's alveolar volume. The processor is further configured to control the gas blender to deliver a second gas over a second portion of the subject's inhalation. The second gas has a partial pressure of gas X equal to a partial pressure of gas X exhaled in a prior breath. The processor is further configured to receive from a sensor an exhaled partial pressure of gas X measured during the subject's exhalation. The processor is further configured to compute the metabolic rate based on the inhaled partial pressure of gas X, the exhaled partial pressure of gas X, and the volume of the first gas.

In some examples, the sensor is configured to measure the subject's tidal volume over a series of breaths, and the processor is further configured to calculate a mean tidal volume based on the measured tidal volumes. The processor then estimates the subject's alveolar volume as the mean tidal volume minus anatomical dead space.

In further examples, the processor is configured to compute the volume of the first gas as about 70% of the estimated alveolar volume.

In further examples, the sensor is configured to measure a characteristic of the series of breaths and the processor is configured to identify a subset of breaths corresponding to a steady state. The subset of breaths is identified by comparing the measured characteristic to a threshold of variability and selecting breaths for which the characteristic is below the threshold of variability. The processor is further configured to determine the mean tidal volume by averaging the tidal volume for the subset of breaths.

In further examples, the sensor is configured to measure the barometric pressure, and the processor is configured to compute the metabolic rate based on the barometric pressure.

In further examples, the processor is configured to compute the metabolic rate by computing

$$\dot{V} X = VA \frac{P E T X - P I X}{PB} ,$$

wherein \dot{V}X represents the metabolic rate of gas X, VA represents the alveolar ventilation, PETX represents the end tidal partial pressure of gas X, PIX represents the inhaled partial pressure of gas X, and PB represents the barometric pressure.

In further examples, gas X comprises oxygen and the processor is configured to compute oxygen consumption (\dot{V}O₂).

In further examples, gas X comprises carbon dioxide and the processor is configured to compute carbon dioxide production (\dot{V}CO₂).

In further examples, gas X comprises both carbon dioxide and oxygen and the processor is configured to compute both \dot{V}CO₂ and \dot{V}O₂.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a schematic diagram showing the sources of carbon dioxide in the average human body.

FIG. 2 is a schematic diagram of a device for measuring metabolic rate using sequential gas delivery.

FIG. 3 is a flowchart of a method for measuring metabolic rate using sequential gas delivery.

FIG. 4 is a schematic diagram of the lung during exemplary performance of the method of FIG. 3.

FIG. 5 is a flowchart of a method for estimating alveolar volume during exemplary performance of the method of FIG. 3.

### DETAILED DESCRIPTION

### Table of Abbreviations

The following abbreviations are used herein:

| | |
|---|---|
| ATP | adenosine triphosphate |
| CO₂ | carbon dioxide |
| FCO₂ | fractional concentration of carbon dioxide |
| FO₂ | fractional concentration of oxygen |
| FECO₂ | fractional concentration of carbon dioxide in an exhaled gas |
| FEO₂ | fractional concentration of oxygen in an exhaled gas |
| FICO₂ | fractional concentration of carbon dioxide in an inhaled gas |
| FIO₂ | fractional concentration of oxygen in an inhaled gas |
| FRC | functional residual capacity |
| G1 | the first gas |
| G2 | the second gas |
| HCO₃_{^{−}} | bicarbonate ion |
| PCO₂ | Partial pressure of carbon dioxide |
| PaCO₂ | arterial partial pressure of carbon dioxide |
| PB | barometric pressure |
| P_{I}X | Inhaled partial pressure of gas X |
| PO₂ | partial pressure of oxygen |
| P_{ET}CO₂ | end-tidal partial pressure of carbon dioxide |
| P_{ET}O₂ | end-tidal partial pressure of oxygen |
| P_{ET}X | end-tidal partial pressure of gas X |
| SGD | sequential gas delivery |
| \dot{V}X | metabolic rate of gas X |
| \dot{V}O₂ | oxygen consumption |
| \dot{V}CO₂ | carbon dioxide production |
| VA | alveolar ventilation |
| \dot{V}Dₐₙₐₜ | anatomical dead space |

### Definitions

The following definitions are used herein:

“About” herein refers to a range of ±20% of the numerical value that follows. In one example, the term “about” refers to a range of ±10% of the numerical value that follows. In one example, the term “about” refers to a range of ±5% of the numerical value that follows.

“Alveolar volume” herein refers to the volume of air or gas that enters the alveoli during respiration.

“Alveolar ventilation” herein refers to the volume of air or gas that contributes to gas exchange.

“Anatomical dead space” is used interchangeably herein with “dead space” to refer to the portion of the respiratory system where gases do not participate in gas exchange with the arterial blood, mostly confined to the trachea, bronchi, and bronchioles.

“Functional residual capacity” herein refers to the volume of air remaining in the lungs at the end of a normal expiration.

“Health condition” herein refers to an anatomical, physiological, or mental state of a subject.

“Metabolic rate” herein refers to the rate at which an organism consumes utilizes oxygen or produces carbon dioxide to sustain vital functions.

### Device and Method

CO₂ is a product of oxidative metabolism where carbon-containing molecules are broken down to atomic carbon and oxidized with two oxygen atoms to form carbon dioxide by the Tricarboxylic acid cycle (also known as the “Krebs cycle” or “citric acid cycle”), breaking down 6-carbon glucose, to two 3-carbon chains, and oxidizing the 3-carbon chains to CO₂ and H₂O, reducing NAD⁺ to NADH₂ and generating ATP. In eukaryotes this takes place inside the mitochondria. The CO₂ thus produced is dissolved in the cellular fluid where it is hydrated into H₂CO₃ (carbonic acid) and an equilibrium is established between hydrogen ion (H⁺) and bicarbonate ion (HCO₃⁻). Accumulating CO₂ molecules increases the partial pressure of CO₂ (PCO₂) which equilibrates across intracellular fluid, extracellular fluid and arterial blood. At the lung, alveolar PCO₂ equilibrates with arterial PCO₂ (PaCO₂) according to the following equations:

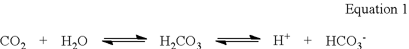

Thus, the pH of blood depends on the concentration of carbon dioxide, as described in the Henderson-Hasselbalch equation:

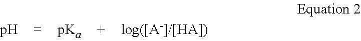

In the Henderson-Hasselbalch equation, pKₐ is the acid base dissociation constant, and [HA] and [A⁻] refer to the equilibrium concentrations of the conjugate acid-base pair used to create the buffer solution.

Oxygen is inhaled into the alveoli and diffuses down its partial pressure gradient into the mixed venous blood entering the alveolar capillaries. The blood circulates through the tissues enabling the oxygen to diffuse into the cells down a concentration gradient and participate in the Kreb's cycle, eventually returning to the lungs to take up additional oxygen. The oxygen taken up by the lung equals the oxygen consumed by the tissues.

The PₐCO₂ is a key determinant of the arterial, and thus, tissue pH. The pH is a major determinant of body enzymatic function. Body enzymes have evolved to be most efficient at a pH of 7.40, with a narrow tolerance of 7.35-7.45. The body tolerates some acidosis but tolerates alkalosis poorly. The body has some ability to compensate for imposed changes in pH. At altitude, people tend to hyperventilate, reducing the PₐCO₂ and thereby increasing the pH. During an extended stay at altitude lasting hours to days, the kidney alters the concentration of strongly dissociated ions like Na⁺ trending the pH towards normal. Renal compensation also occurs for respiratory acidosis. In addition, respiratory compensation occurs for renal acid-base disequilibria: hypoventilation for alkalosis and hyperventilation of acidosis.

At a basal resting state, the oxygen consumption (\dot{V}O₂) and CO₂ production (\dot{V}CO₂) are constant and reflect the biochemical body processes and heat production.

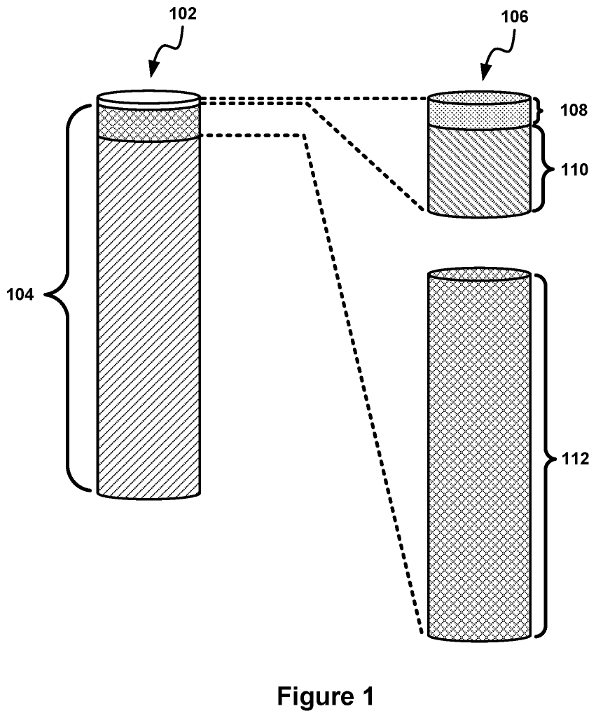

FIG. 1 is a schematic diagram showing where CO₂ is stored in an average human body. 102 represents the body's entire store of CO₂. CO₂ in the lungs 108 and blood 110 comprise a pool of CO₂ that is rapidly exchanged 106. CO₂ in the alveolar gas and in lung tissue 108 comprises about 0.2 L of CO₂. The blood 110 contains about 2.7 L of CO₂: about 80% is in the form of H⁺ and HCO₃⁻; about 5-10% is dissolved in plasma and interstitial fluid; and about 5-10% is combined with hemoglobin as carbamino hemoglobin.

The bones contain carbonates which are fixed and not part of the CO₂ exchange. Bone carbonate 104 comprises about 120 L of CO₂. The bones also contain bicarbonate (HCO₃⁻) 112 which is in equilibrium with blood HCO₃⁻. This exchangeable bicarbonate represents about 9 L of CO₂ and participates in the daily production of CO₂. In total, the exchangeable pool of CO₂ (represented in FIG. 1 at 108, 110, and 112) is about 14 L and acts as a large buffer pool.

The measure of \dot{V}CO₂ is traditionally performed by collecting the timed volume of exhaled CO₂, by, for example collecting exhaled gas for a period of time into a large collection bag. The total volume of collected gas, and its CO₂ concentration is used to compute the volume of exhaled CO₂. Dividing this volume by the collection time, will result in a \dot{V}CO₂ measure.

The present invention will be described with respect to the figures herein.

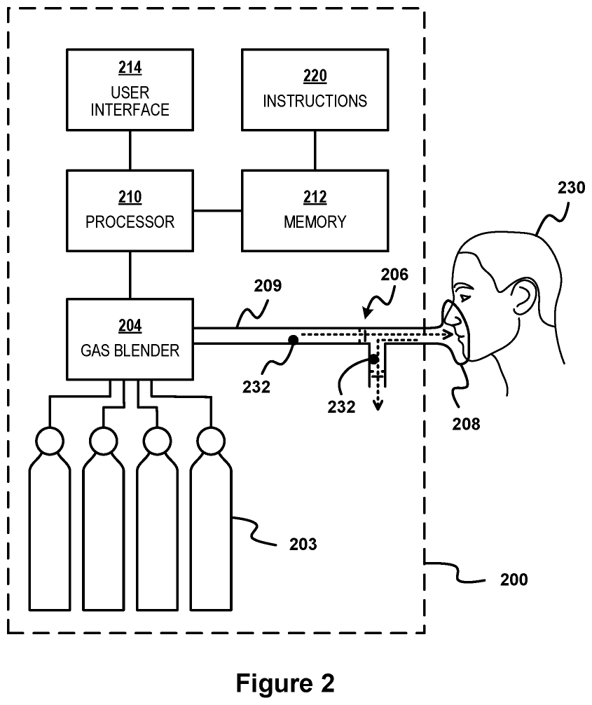

FIG. 2 shows a device 200 for measuring metabolic rate using sequential gas delivery.

FIG. 2 shows a device 200 for measuring metabolic rate. The device 200 is configured to provide sequential gas delivery to a subject 230 and target a PaO₂ while maintaining normocapnia. The device 200 includes gas supplies 203, a gas blender 204, a mask 208, a processor 210, memory 212, and a user interface 214. The device 200 may be configured to control end-tidal PCO₂ and end-tidal PO₂ by generating predictions of gas flows to actuate target end-tidal values. The device 200 may be an RespirAct™ device, made by Thornhill Medical™ of Toronto, Canada, specifically configured to implement the techniques discussed herein. For further information regarding sequential gas delivery, U.S. Pat. No. 8,844,528, US Publication No. 2018/0043117, and U.S. Pat. No. 10,850,052, which are incorporated herein by reference, may be consulted.

The gas supplies 203 may provide carbon dioxide, oxygen, nitrogen, and air, for example, at controllable rates, as defined by the processor 210. A non-limiting example of the gas mixtures provided in the gas supplies 203 is:
Gas A: 10% oxygen (O₂), 90% nitrogen (N₂);
Gas B: 10% oxygen (O₂), 90% carbon dioxide (CO₂);
Gas C: 100% oxygen (O₂); and
Calibration gas: 10% (O₂), 9% carbon dioxide (CO₂), 81% nitrogen (N₂).

The gas blender 204 is connected to the gas supplies 203, receives gases from the gas supplies 203, and blends received gases as controlled by the processor 210 to obtain a gas mixture, such as a first gas (G1) and a second gas (G2) for sequential gas delivery. The gas blender 204 may comprise one or more mass flow controllers for monitoring the flow of gas from the gas supplies 203. The mass flow controller may comprise a device that measures the mass flow rate from the gas supplies 203 as an amount of gas passing through the controller per unit of time. The gas blender 204 may further include one or more variable orifices for controlling the flow of gases from the gas supplies 203. The variable orifice may comprise an opening from the gas supplies 203 which is adjustable in size to control the flow rate of gas from the gas supplies 203 to the subject 230. In certain embodiments, the gas blender 204 controls the variable orifice in response to measurements of flow rate obtained by the mass flow controller.

The first gas (G1) comprises an inhaled partial pressure of gas X. In some examples, the first gas (G1) may have a composition similar to atmospheric air (which typically comprises about 0.04% carbon dioxide, 21% oxygen, and 79% nitrogen). In some examples, the first gas (G1) comprises atmospheric air and the gas blender 204 is not required to provide the first gas (G1). In further examples, the concentration of carbon dioxide is 0% or about 0%.

The second gas (G2) is a neutral gas in the sense that it has about the same partial pressure of gas X as an end-tidal gas exhaled by the subject 230 in a prior breath (PETX).

In some examples, the second gas (G2) may include gas actually exhaled by the subject 230 (referred to herein as a “rebreathed gas”). In these examples, the device 200 may further include a rebreathing compartment (not shown) for receiving the rebreathed gas from a prior breath via a conduit and providing the rebreathed gas to the subject 230 via the conduit. The rebreathing compartment may comprise a valve for controlling when the rebreathed gas is provided to the subject 230.

For the purposes of measuring metabolic rate, gas X may be either O₂ or CO₂. Particular examples described herein may refer to either oxygen or carbon dioxide, however it should be understood that the method may be similarly applied to carbon dioxide or oxygen, or both carbon dioxide and oxygen.

The processor 210 may control the gas blender 204, such as by electronic valves, to deliver the first and second gases in a controlled manner.

The mask 208 is connected to the gas blender 204 and delivers gas to the subject 230. The mask 208 may be sealed to the subject's face to ensure that the subject only inhales gas provided by the gas blender 204 to the mask 208. In some examples, the mask is sealed to the subject's face with skin tape such as Tegaderm™ (3M, Saint Paul, Minnesota). A valve arrangement 206 may be provided to the device 200 to limit the subject's inhalation to gas provided by the gas blender 204 and limit exhalation to the room. In the example shown, the valve arrangement 206 includes an inspiratory one-way valve from the gas blender 204 to the mask 208, a branch between the inspiratory one-way valve and the mask 208, and an expiratory one-way valve at the branch. Hence, the subject 230 inhales gas from the gas blender 204 and exhales gas to the room.

The gas supplies 203, gas blender 204, and mask 208 may be physically connectable by a conduit 209, such as tubing, to convey gas. Any suitable number of sensors 232 may be positioned at the gas blender 204, mask 208, and/or conduits 209 to measure flow rate, composition, pressure, temperature, and/or similar properties of gases and provide these measurements to the processor 210. Gas properties may be sensed at any suitable location, so as to measure properties of gas inhaled or exhaled by the subject 230. In particular embodiments, the sensors 232 include a sensor to measure the composition and volume of gas exhaled by the subject 230 and a sensor to measure the composition and volume of gas inhaled by the subject 230. In further embodiments, the sensor 232 measures the barometric pressure (PB). One or more sensors 232 may be located apart from the gas blender 204 and conduits 209 so as to measure the air pressure and temperature of the room.

The processor 210 may include a central processing unit (CPU), a microcontroller, a microprocessor, a processing core, a field-programmable gate array (FPGA), an application-specific integrated circuit (ASIC), or a similar device capable of executing instructions. The processor 210 may be connected to and cooperate with the memory 212 that stores instructions and data.

The memory 212 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface 214 may include a display device, touchscreen, keyboard, speaker, buttons, the like, or a combination thereof to allow for operator input and/or output. Although not shown in FIG. 2, the device 200 may further include a network interface for receiving data via a network.

Instructions 220 may be provided to carry out the functionality and methods described herein. The instructions 220 may be directly executed, such as a binary file, and/or may include interpretable code, bytecode, source code, or similar instructions that may undergo additional processing to be executed. The instructions 220 may be stored in the memory 212.

The gas supplies 203, gas blender 204, processor 210, memory 212, user interface 214, sensors 232, and instructions 220 are not necessarily be housed in a single device. In some examples, the processor 210 is connected to the gas blender 204 and sensors 232 via a network and transmits instructions to the gas blender 204 and sensors 232 via the network.

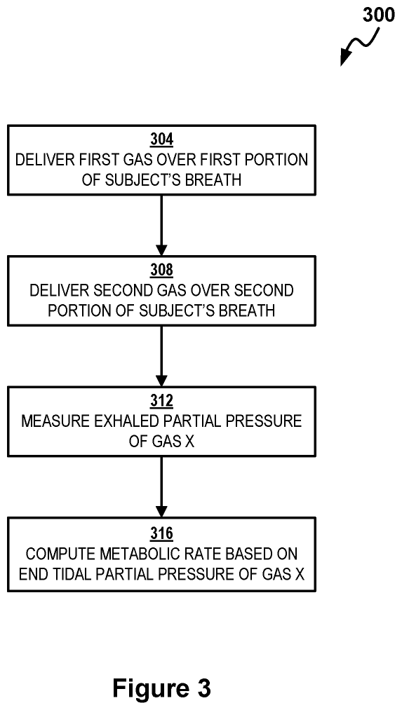

FIG. 3 shows a method 300 of measuring a subject's metabolic rate using sequential gas delivery. In the embodiments described herein, method 300 is performed on device 200.

Block 304 comprises delivering a first gas over a first portion of the subject's inhalation. As part of block 304, the gas blender 204 delivers the first gas to the subject 203 via the conduit 209. In the embodiments described herein, block 304 is performed by processor 210 which controls the gas blender to deliver the first gas. The first gas comprises an inhaled partial pressure of gas X. Gas X is generally O₂ or CO₂ or both O₂ and CO₂. In some embodiments, the inhaled partial pressure of gas X is a pre-determined value retrieved from the memory 212. In other examples, the inhaled partial pressure of gas X in the first gas is entered at a user interface 214 or received via a network.

Block 308 comprises delivering a second gas over a second portion of the subject's inhalation. As part of block 308, the gas blender 204 may provide the second gas to the subject via the conduit 209. In embodiments where the second gas comprises a rebreathed gas, the second gas is provided instead by a rebreathing compartment.

The second gas comprises a partial pressure of gas X that is equal or approximately equal to an end-tidal partial pressure of gas X exhaled in a prior breath. Since the end-tidal gas is equilibrated with the blood, the partial pressure of gas X in the second gas is equal or approximately equal to the arterial partial pressure of gas X. In some examples, the volume of the second gas comprises the remainder of the subject's breath. Generally, the second gas displaces the first gas from the subject's dead space into the alveoli so that all or essentially all of the first gas is delivered to the alveoli.

Since the volume of the first gas is less than the subject's alveolar volume, at least a portion of the second gas will enter the subject's alveoli, however the composition of the second gas is equal to the arterial partial pressure of CO₂ and O₂, and therefore does not participate in gas exchange. Thus, blocks 304 and 308 enable the device 200 to deliver a known amount of gas X to the subject's arterial blood.

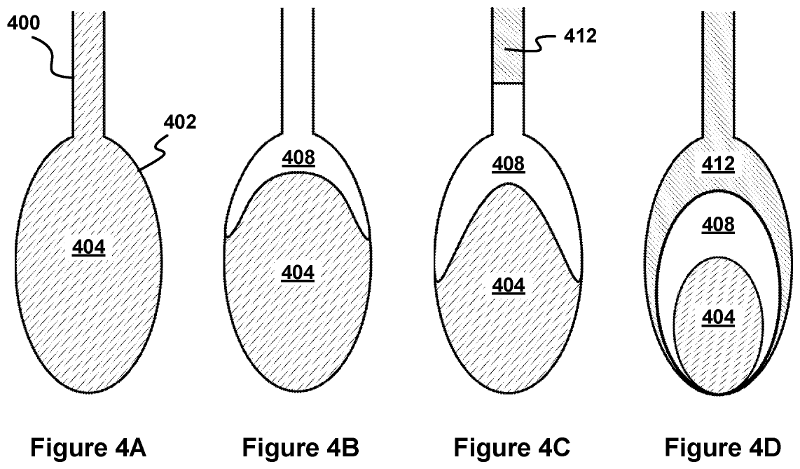

Exemplary performance of sequential gas delivery, as described in blocks 304 and 308, is illustrated in FIGS. 4A to 4D. FIGS. 4A to 4D are schematic diagrams of a lung, which includes the anatomical dead space 400 and the alveoli 402. The pulmonary arteries undergo exchange with gases in the alveoli 402, but gases in the anatomical dead space 400 do not participate in gas exchange. As shown in FIG. 4A, the lung contains the functional residual capacity (FRC) before the subject 230 begins to inhale. Over a first portion of the breath, the processor 210 controls the gas blender 204 to deliver the first gas 408, as shown in FIG. 4B. Over a second portion of the breath, the processor 210 controls the gas blender 204 to deliver the second gas 412, as shown in FIG. 4C. Since the volume of the first gas is less than the alveolar volume, the second gas 412 displaces the first gas from the anatomical dead space 400, as shown in FIG. 4D. The first gas 408 equilibrates with the arterial blood, but the second gas 412 is neutral and therefore does not affect or minimally affects the concentration of gas X in the pulmonary arterial blood.

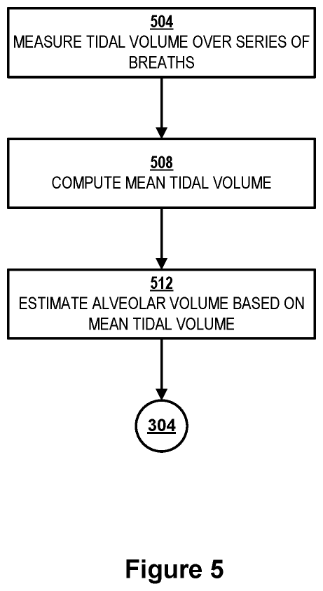

Since the volume of the first gas is less than the subject's alveolar volume, the device 200 may estimate the subject's alveolar volume before performing block 304. In some examples, the device 200 estimates the subject's alveolar volume when breathing at rest before performing block 304. In some examples, the alveolar volume is estimated based on the mean tidal volume of the subject 230, as shown in FIG. 5.

Block 504 comprises measuring the subject's tidal volume over a subset of breaths. In device 200, block 504 is performed by the sensors 232 which measure the tidal volume as the subject inhales and exhales. During performance of block 504, the subject may be coached to breathe at a steady state.

Block 508 comprises computing a mean tidal volume. In device 200, block 508 is performed by the processor 210 which receives the measurements obtained at block 504 from the sensors 232 and computes the mean tidal volume based on the measurements.

In some examples, the processor 210 computes the mean tidal volume by averaging the tidal volume measured for the breaths in the series of breaths. In other examples, the processor 210 identifies a subset of breaths from the series of breaths corresponding to a steady state and computes the mean tidal volume by averaging the tidal volume for the subset of breaths. The processor 210 may select the subset of breaths by measuring at least one characteristic of the series of breaths, calculating the variability of the measured characteristic among the series of breaths, and selecting a subset of breaths according to a threshold variability. Generally, the measured characteristic for the subset of breaths is below the threshold of variability. The measured characteristic may include but is not limited to breath rate, tidal volume, minute ventilation, P_{ET}CO₂, P_{ET}O₂, or a combination thereof. In certain examples, the threshold variability is 5%. In some examples, the threshold variability is 10%. In other examples, the threshold variability is 15%. In further examples, the threshold variability is 20%. In some examples, the processor 210 is configured to choose consecutive breaths as the subset of breaths. In one specific, non-limiting example, the processor 210 selects the subset of breaths as a plurality of breaths having a standard deviation of tidal volume that is less than 10% of the mean of the tidal volumes.

Block 512 comprises estimating the subject's alveolar volume. In device 200, block 512 is performed by processor 210 which estimates the alveolar volume based on the mean tidal volume computed at block 508. The relevant variables for estimating the alveolar volume may be retrieved from memory 212, received via the network, or received at the user interface 214. In some examples, the alveolar volume is further determined based on one or more physiological parameters such as age, body weight, height, biological sex, and disease condition. In particular examples, the alveolar volume is estimated as mean tidal volume minus anatomical dead space. In some examples, anatomical dead space in healthy adults is estimated to be about 2 mL/kg of body weight. In other examples, anatomical dead space in a healthy adult is estimated to be about ⅓ of the mean tidal volume. In further examples, alveolar volume in a healthy adult is estimated to be 70% of the mean tidal volume. The anatomical dead space is greater in children and smaller when the subject is in supine position.

The method 500 of FIG. 5 for estimating alveolar volume is not strictly necessary, and the alveolar volume may be measured or estimated according to alternative methods.

A skilled person will appreciate that, when there is certainty of the volume of the first gas (G1) entering the alveoli, an accurate estimate of the tidal volume is not strictly necessary. The volume of G1 entering the alveoli and available for gas exchange and the end-tidal partial pressure of the gases being measured are the crucial measurements required. If the volume of G1 is inadvertently underestimated, the measured \dot{V}CO₂ and \dot{V}O₂ will be underestimated. Similarly if the volume of G1 is inadvertently overestimated, the \dot{V}CO₂ and \dot{V}O₂ will be overestimated.

Thus, in preferred embodiments, the processor 210 underestimates the alveolar volume or delivers a volume of the first gas that is smaller than the estimated alveolar volume, so as to increase the probability that all of the first gas is delivered to the alveoli. In specific examples, the volume of the first gas is about or less than 90% of the estimated alveolar volume. In other examples, the volume of the first gas is about or less than 80% of the estimated alveolar volume. In further examples, the volume of the first gas is about or less than 70% of the estimated alveolar volume. In yet further examples, the volume of the first gas is about or less than 60% of the estimated alveolar volume. In yet further examples, the volume of the first gas is about or less than 50% of the estimated alveolar volume. In yet further examples, the volume of the first gas is about or less than 40% of the estimated alveolar volume. Generally, there is a lower limit to the volume of the first gas, which depends on the subject's metabolic rate. If the volume of the first gas is too small, carbon dioxide will accumulate in the subject, preventing the subject from reaching a steady state. As a result, the \dot{V}O₂ and \dot{V}CO₂ will be underestimated.

In some embodiments, the subject 230 is at rest during performance of blocks 304 and 308, however it is not strictly necessary for the subject 230 to be at rest. In some examples, the subject 230 is performing a physical or mental activity.

Block 312 comprises measuring an exhaled partial pressure of gas X during the subject's exhalation. In device 200, block 312 may be performed by one or more of the sensors 232 which measure the partial pressure of gas X in the subject's exhalation. In particular embodiments, the sensors 232 measure the exhaled partial pressure of gas X at the end of the exhalation (referred to herein as the “end-tidal partial pressure of gas X” or “PETX”). As part of block 312, one or more of the sensors 232 may further measure the barometric pressure (PB). As part of block 312, the sensors 232 transmit the measurements to the processor 210. As a further part of block 312, the sensors 232 may transmit the measurements to the memory 212.

Block 316 comprises computing the metabolic rate based on the volume of the first gas delivered at block 304, the inhaled pressure of gas X delivered at block 304, and the exhaled partial pressure of gas X measured at block 312. In device 200, block 316 is performed by processor 210 which receives the measurements from the sensors 232 and computes the metabolic rate accordingly.

In some examples, gas X is oxygen, and the metabolic rate is the oxygen consumption rate (\dot{V}O₂). In other examples, gas X is carbon dioxide, and the metabolic rate is the carbon dioxide production rate (\dot{V}CO₂).

As part of block 316, the processor 210 may convert the exhaled partial pressure of gas X to a fractional concentration of gas X. PCO₂ and PO₂ are the partial pressures of CO₂ and O₂ respectively. To convert PCO₂ and PO₂ to fractional concentrations, the processor 210 divides the partial pressure of the gas X by the atmospheric barometric pressure (PB). The barometric pressure may be measured by a sensor, received as an input at the user interface 214, retrieved from the memory 212, or received via a network. The fractional volumes may be calculated according to Equations 3 and 4:

$${F CO}_{2} = {P CO}_{2} / PB \qquad Equation 3$$

$${F O}_{2} = {P O}_{2} / PB \qquad Equation 4$$

To calculate carbon dioxide production, the processor 210 multiplies the alveolar ventilation (VA) by the difference between the fractional pressure of the expired CO₂ (FECO₂) and the fractional pressure of the inspired CO₂ in the first gas (FICO₂), as shown in Equation 5:

$$\dot{V} {CO}_{2} = VA ( {FE CO}_{2} - {FI CO}_{2} ) \qquad Equation 5$$

Since the second gas is neutral with respect to the partial arterial pressure of gas CO₂, the alveolar ventilation (VA) is the volume of the first gas, which was imposed at block 304. When breathing atmospheric air, FICO₂ may be estimated as zero.

To calculate oxygen consumption, the processor 210 similarly multiplies the alveolar ventilation (VA) by the difference between the fractional pressure of inspired oxygen in the first gas (FIO₂) and the fractional pressure of expired oxygen (FEO₂), as shown in Equation 6:

$$\dot{V} {O}_{2} = VA ( {FI O}_{2} - {FE O}_{2} ) \qquad Equation 6$$

Since the second gas is neutral with respect to the partial arterial pressure of gas O₂, the effective alveolar ventilation (VA) is the volume of the first gas, which was imposed at block 304.

A simplified equation by which the processor 210 may compute the metabolic rate is:

$$\dot{V} X = V A \frac{PETX - P I X}{PB} \qquad Equation 7$$

In Equation 7, \dot{V}X represents the metabolic rate of gas X, VA represents the alveolar ventilation (the volume of the first gas (G1)), PETX represents the end-tidal partial pressure of gas X, PIX represents the inhaled partial pressure of gas X in the first gas, and PB represents the barometric pressure.

For improved accuracy, blocks 304 to 312 may be repeated for a plurality of breaths to obtain a plurality of measurements. As part of block 316, the processor 210 may statistically analyze the plurality of measurements to compute the metabolic rate. In some examples, the metabolic rate is based on the mean, median, or mode of the plurality of measurements. In some examples, the processor 210 removes outliers from the plurality of measurements before computing the metabolic rate. In some examples, the processor 210 identifies a subset of the plurality of breaths, the subset corresponding to steady state breathing, and computes the metabolic rate based on only the measurements obtained from the subset of breaths. The processor 210 may select the subset of breaths by measuring at least one characteristic of the series of breaths, calculating the variability of the measured characteristic among the series of breaths, and selecting a subset of breaths according to a threshold variability. Generally, the measured characteristic for the subset of breaths is below the threshold of variability. The measured characteristic may include but is not limited to breath rate, tidal volume, minute ventilation, P_{ET}O₂, P_{ET}CO₂, or a combination thereof. In certain examples, the threshold variability is 5%. In some examples, the threshold variability is 10%. In other examples, the threshold variability is 15%. In further examples, the threshold variability is 20%. In some examples, the processor 210 is configured to choose consecutive breaths as the subset of breaths. In one specific, non-limiting example, the processor 210 selects the subset of breaths as a plurality of breaths having a standard deviation of tidal volume that is less than 10% of the mean of the tidal volumes.

As part of block 316, the processor 210 may control the user interface 214 to output the metabolic rate at a display. In some examples, the metabolic rate is stored in the memory 212.

The processor 210 may further interpret the metabolic rate according to specific applications, examples of which are provided herein.

Metabolic rate can be used to assess various health conditions such as hypothyroidism and hypothyroidism. After computing the metabolic rate at block 316, the processor 210 may be further configured to compare the subject's metabolic rate to a pre-determined threshold, the pre-determined threshold indicating a likelihood that the subject has a health condition. In a particular example, an elevated metabolic rate may indicate hyperthyroidism while a lower metabolic rate could suggest hypothyroidism. If the metabolic rate is higher or lower than the pre-determined threshold, the processor 210 may control the user interface 214 to display an alert showing the disease condition. The alert may further show the threshold, the metabolic rate, other clinical indications of the health condition, a recommended treatment plan, or a combination thereof.

Metabolic rate may be used in weight management. After computing the metabolic rate at block 316, the processor 210 may be further configured to estimate the subject's daily caloric intake and expenditure and control the user interface 214 to display an alert which includes the daily caloric intake and expenditure. According to the subject's weight goal, which may be input at the user interface 214, received via a network, or retrieved from the memory 212, the processor 210 may further generate a nutrition plan based on the daily caloric intake and expenditure and the subject's weight goal and control the user interface 214 to output the nutrition plan. The subject's weight goal may be to maintain, lose, or gain weight.

Metabolic rate may be used in athletics to optimize physical performance. After the metabolic rate is computed at block 316, the processor 210 may be further configured to generate a training program or nutrition plan for the subject based on the metabolic rate. Generating the training program or nutrition plan may be further based on parameters input at the user interface 214, received via a network, or retrieved from the memory 212. Such parameters may include but are not limited to a training goal, body weight, height, biological sex, and the like.

Metabolic rate may also be used in personalized medicine to select a treatment for an appropriate treatment for a disease condition. After the metabolic rate is computed at block 316, the processor 210 may be further configured to generate a treatment plan for the subject based on the metabolic rate. The treatment plan may include, but is not limited to, one or more therapeutic agents, a dose, a dose regimen, or a combination thereof. Generating the treatment plan may be further based on parameters input at the user interface 214, received via a network, or retrieved from the memory 212. Such parameters may include but are not limited to disease condition, age, biological sex, height, weight, known allergies, genetics, comorbidity, lifestyle factors, environmental factors, mental health conditions, and combinations thereof.

In view of the above, it will now be apparent that variants, combinations, and subsets of the foregoing embodiments are contemplated. For example, while method 300 was discussed above in relation to a human subject, other animals are contemplated.

It will now be apparent to a person of skill in the art that the present specification affords certain advantages over the prior art. In comparison to the prior art, the method and device described above are more accurate and precise because they do not require pneumotachographs, measuring tidal volume, integrating the area under PCO₂ curves, integrating the area under PO₂ curves, consistent tidal volume, or consistent breathing frequency. Currently known measures of metabolic rate rely on measuring flow and tidal volumes in order to measure alveolar ventilation. This requires integration of signals of partial pressures of CO₂ and PO₂ to measure volumes of these gases.

In contrast, the presently disclosed method and device does not measure alveolar ventilation, rather it imposes alveolar ventilation. As a benefit for this strategy, it is not strictly necessary to guess the alveolar ventilation when tidal volumes overlap with the anatomical dead space. The device can use sequential gas delivery methods to precisely and accurately set the alveolar ventilation by delivering controlled volumes of gases.

To know the alveolar ventilation (VA) precisely, the device starts by generating a precise volume of the first gas. Since variable orifice and mass flow controllers are highly precise and accurate, the alveolar ventilation (VA) can be very precisely set and accurately known. The accuracy of VA comes, not from accurate measurements, but from the precision of the flow control instruments. Consequently, the partial pressure of CO₂ (PCO₂) does not have to be measured over time and integrated, which introduces errors. Rather, only the end-tidal PCO₂ (P_{ET}CO₂) and barometric pressure (PB) need be measured. When the P_{ET}CO₂ is measured from exhaled gas over prolonged sampling, it too can be performed very accurately and precisely with readily available CO₂ sensors. This yields high precision and accuracy in measuring \dot{V}CO₂. Similarly, \dot{V}O₂ can be measured accurately from accurate alveolar ventilation (VA), according to the following equation: \dot{V}O₂=VA(FIO₂−FEO₂).

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

1. A method of measuring a subject's metabolic rate using sequential gas delivery, the method comprising:
delivering a first gas over a first portion of the subject's inhalation, the first gas comprising an inhaled partial pressure of gas X, wherein the volume of the first gas is equal to or less than the subject's alveolar volume;
delivering a second gas over a second portion of the subject's inhalation, the second gas comprising a partial pressure of gas X equal to an end-tidal partial pressure of gas X exhaled in a prior breath;
measuring an exhaled partial pressure of gas X during the subject's exhalation; and
computing the metabolic rate based on the inhaled partial pressure of gas X, the exhaled partial pressure of gas X, and the volume of the first gas.

2. The method of claim 1 further comprising:
measuring the subject's tidal volume over a series of breaths;
calculating a mean tidal volume based on the measured tidal volumes; and
estimating the subject's alveolar volume as the mean tidal volume minus anatomical dead space.

3. The method of claim 2 wherein the volume of the first gas is computed as about 70% of the estimated alveolar volume.

4. The method of claim 3 further comprising identifying a subset of breaths corresponding to a steady state;
wherein the subset of breaths is identified by comparing a measured characteristic for the subset of breaths to a threshold of variability and selecting breaths for which the characteristic is below the threshold of variability; and
wherein calculating the mean tidal volume comprises averaging the tidal volume for the subset of breaths.

5. The method of claim 1 further comprising measuring the barometric pressure, wherein computing the metabolic rate is further based on the barometric pressure.

6. The method of claim 5 wherein computing the metabolic rate comprises computing
$$\dot{V} X = VA \frac{P E T X - P I X}{PB}$$
wherein \dot{V}X represents the metabolic rate of gas X, VA represents the volume of the first gas, PETX represents the end tidal partial pressure of gas X, PIX represents the inhaled partial pressure of gas X, and PB represents the barometric pressure.

7. The method of claim 6 wherein gas X comprises oxygen and computing the metabolic rate comprises computing the oxygen consumption rate (\dot{V}O₂).

8. The method of claim 6 wherein gas X comprises carbon dioxide and computing the metabolic rate comprises computing the carbon dioxide production rate (\dot{V}CO₂).

9. The method of claim 6 wherein gas X comprises both carbon dioxide and oxygen and computing the metabolic rate comprises computing both \dot{V}CO₂ and \dot{V}O₂.

10. A non-transitory computer-readable medium comprising instructions that, when executed by a processor, perform the method of claim 1.

11. A device for measuring a subject's metabolic rate comprising:
a gas blender; and
a processor connected to the gas blender;
wherein the processor is configured to:
control the gas blender to deliver a first gas over a first portion of the subject's inhalation, the first gas comprising an inhaled partial pressure of gas X, wherein the volume of the first gas is equal to or less than the subject's alveolar volume;
control the gas blender to deliver a second gas over a second portion of the subject's inhalation, the second gas comprising an end-tidal partial pressure of gas X equal to a partial pressure of gas X exhaled in a prior breath;
receive from a sensor an exhaled partial pressure of gas X measured during the subject's exhalation; and
compute the metabolic rate based on the inhaled partial pressure of gas X, the exhaled partial pressure of gas X, and the volume of the first gas.

12. The device of claim 11,
wherein the sensor is configured to measure the subject's tidal volume over a series of breaths; and
wherein the processor is further configured to:
calculate a mean tidal volume based on the measured tidal volumes; and
estimate the subject's alveolar volume as the mean tidal volume minus anatomical dead space.

13. The device of claim 12 wherein the processor is configured to compute the volume of the first gas as about 70% of the estimated alveolar volume.

14. The device of claim 13,
wherein the sensor is configured to measure a characteristic of the series of breaths;
wherein the processor is configured to identify a subset of breaths corresponding to a steady state;
wherein the subset of breaths is identified by comparing the measured characteristic to a threshold of variability and selecting breaths for which the characteristic is below the threshold of variability; and
wherein the processor is configured to determine the mean tidal volume by averaging the tidal volume for the subset of breaths.

15. The device of claim 11,
wherein the sensor is configured to measure the barometric pressure; and
wherein the processor is configured to compute the metabolic rate based on the barometric pressure.

16. The device of claim 15 wherein the processor is configured to compute the metabolic rate by computing
$$\dot{V} X = VA \frac{P E T X - P I X}{P B} ,$$
wherein \dot{V}X represents the metabolic rate of gas X, VA represents the volume of the first gas, PETX represents the end tidal partial pressure of gas X, PIX represents the inhaled partial pressure of gas X, and PB represents the barometric pressure.

17. The device of claim 16 wherein gas X comprises oxygen and the processor is configured to compute oxygen consumption (\dot{V}O₂).

18. The device of claim 16 wherein gas X comprises carbon dioxide and the processor is configured to compute carbon dioxide production (\dot{V}CO₂).

19. The device of claim 16 wherein gas X comprises both carbon dioxide and oxygen and the processor is configured to compute both \dot{V}CO₂ and \dot{V}O₂.
